(19) United States
(12) Patent Application Publication
(10) Pub. No.: US 2020/0230325 A1
(43) Pub. Date: Jul. 23, 2020

(54) CARTRIDGE SYSTEM FOR A DRUG DELIVERY DEVICE

(71) Applicant: Novo Nordisk A/S, Bagsvaerd (DK)

(72) Inventors: Henrik Bengtsson, Taastrup (DK); Lars Korsbjerg Nielsen, Birkeroed (DK); Bo Kvolsbjerg, Helsingoer (DK)

(21) Appl. No.: 16/650,584

(22) PCT Filed: Sep. 28, 2018
(86) PCT No.: PCT/EP2018/076519

§ 371 Date: Mar. 25, 2020

(30) Foreign Application Priority Data
Sep. 30, 2017 (EP) 17194273.3
Oct. 2, 2017 (EP) 17194481.2

(51) Int. Cl. A61M 5/315; A61M 5/24

(52) U.S. Cl. CPC A61M 5/31568; A61M 5/2422; A61M 5/31513; A61M 2205/3306; A61M 2207/00; A61M 2205/3553; A61M 2205/50; A61M 2205/3584; A61M 2205/3375

(57) ABSTRACT

A cartridge system for use with a drug delivery device (100′), comprising: —a pre-filled cartridge comprising: a) a cartridge body (210) accommodating a drug, b) a piston (250) slidably disposed in the cartridge body (210) and configured for providing a proximal seal, the piston (250) comprising: b1) a piston first member (260) comprising a first material and forming a distal end, a proximal end and a sealing periphery in contact with the cartridge body (210), wherein the distal end is in contact with the drug and wherein the proximal end of the piston first member (260) includes a proximally facing opening, and b2) a piston second member (270) comprising a material of lower compressibility compared to the first material, the piston second member (270) comprising a sleeve-formed portion arranged in the proximally facing opening of the piston first member (260), wherein the piston second member (270) comprises a proximally facing opening forming a socket; and—a plug unit (280) coupleable or coupled into the socket of the piston second member (270), wherein the plug unit (280) comprises an electronic sensor unit (290) configured to determine the axial position of the piston (250) within the cartridge body (210) and wherein the plug unit (280) is at least partially received within the proximally facing opening of the piston first member (260).

The present invention relates to a cartridge system for a drug delivery device and a method for capturing drug delivery dose data. In particular, the invention addresses the issue of providing an electronic data capturing system for and in a drug delivery device.

### BACKGROUND

In the disclosure of the present invention reference is mostly made to the treatment of diabetes by delivery of insulin, however, this is only an exemplary use of the present invention.

Drug injection devices have greatly improved the lives of patients who must self-administer drugs and biological agents. Drug injection devices may take many forms, including simple disposable devices that are little more than an ampoule with an injection means or they may be durable devices adapted to be used with replaceable pre-filled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, from the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of ejection information from medication delivery systems. Thus, a number of injection devices with a dose monitoring/acquisition feature has been provided, see e.g. in US 2009/0318865, WO 2010/052275 which both show examples of injection devices with integrated dose logging arrangements. Typically, such arrangements are mainly suitable when designing durable devices.

Disposable devices require production costs to be low, since they have a very limited lifetime. Furthermore, the environmental impact of implementing traditional electronic circuitry in disposable devices limits the possibilities of practical use of such logging devices in disposable devices.

Correspondingly, data acquisition/monitoring functionality have been proposed to be provided in a separate device to be put on or in the injection device, i.e. some kind of accessory e.g. an add-on module to the injection device. For example, WO 2010/098927 and WO 2010/128493 each discloses a medical module which is configured to be attached to a drug delivery pen, the module being adapted to detect and store selected and ejected dosages as well as other data.

WO 2014/128155 discloses an electronic logging unit for a drug delivery device where the logging unit is accommodated within the barrel of a held drug-filled cartridge at the open proximal end thereof, i.e. arranged between the piston of the cartridge and the piston rod of the expelling mechanism. Ejection information is provided in a simple manner by detecting the amount of relative rotation of the threaded piston rod. Monitoring movements of the piston rod potentially eliminates many tolerance issues typically associated with add-on modules that attaches to the exterior of a drug delivery device. However, the solution with the logging unit arranged between the piston and the piston rod adds to the length of the drug delivery device. Further examples of systems incorporating a sensor arranged at a rear portion of a piston are disclosed in WO 2014/009442, WO2014/053493, WO 2014/067879 and WO 2014/118107.

US 2015/174342 discloses a dosage control system for a syringe wherein a transducer located in or on the plunger is used for determining an amount of medication administered from the syringe. US 2003/233075 and U.S. Pat. No. 9,623,191 disclose different kinds of sensors arranged in a piston body. Further, WO 2013/064590 discloses a two-member piston for a cartridge wherein an electronic circuit is arranged at the distal portion of a piston, more specifically at the interface region between first and second piston members.

Requirements of clean-room operations and sterilisation using steam present problems if sensor electronics and batteries are to be fitted in the cartridge assembly prior to cartridges being steamed and leaving the clean-room production facility. Further, since any electronic components, circuitry, sensors and power sources themselves will add to the costs, electronic logging of use may be desired in some markets, while in other markets a lower price of the device is preferred. Aiming at marketing different variants increases manufacturing and logistic costs.

Having regard to the above, it is an object of the present invention to provide a cartridge system and a method of forming such system wherein one or more of the shortcomings of the prior art solutions are avoided.

### SUMMARY

It is an object of the present invention to provide a medical drug injection device featuring improved needle safety. It is a further object of the invention to provide a simplified and robust design of such medicament injector.

In the disclosure of the present invention, embodiments and aspects will be described which will address one or more of the above objects or which will address objects apparent from the below disclosure as well as from the description of exemplary embodiments.

In a first aspect, the present invention relates to a cartridge system for use with a drug delivery device, wherein the cartridge system comprises:
a pre-filled cartridge comprising:
a cartridge body accommodating a drug,
a piston slidably disposed in the cartridge body and configured for providing a proximal seal, the piston comprising:
   a piston first member comprising a first material and forming a distal end, a proximal end and a sealing periphery in contact with the cartridge body, wherein the distal end is in contact with the drug and wherein the proximal end of the piston first member includes a proximally facing opening, and
   a piston second member comprising a material of lower compressibility compared to the first material, the piston second member comprising a sleeve-formed portion arranged in the proximally facing opening of the piston first member, wherein the piston second member comprises a proximally facing opening forming a socket; and
a plug unit coupleable or coupled into the socket of the piston second member, wherein the plug unit comprises an electronic sensor unit configured to determine the axial position of the piston within the cartridge body. The piston with the plug unit coupled into the socket of the piston second member defines a piston assembly wherein the plug unit is at least partially received within the proximally facing opening of the piston first member, and wherein the piston assembly defines a proximally arranged thrust receiving surface configured for receiving a thrust force from a piston rod of the drug delivery device.

From a measuring perspective for determining expelling information, the best position to place sensors and electronics is considered to be in the prefilled cartridge containing the drug, since this will enable exclusion of all influences of tolerances. By forming the plug unit to be received at least partly within the proximally facing opening of the piston first member a comparatively rigid part of the piston assembly will mechanically couple with the electronic sensor unit and enable position detection at a location which is in close proximity with the distal end of the piston first member being is in contact with the drug. This enables position detection of the distal end of the piston first member with increased accuracy.

At the same time the electronic sensor unit can be added on to the cartridge itself during final assembly. The alternative would require handling two different cartridges of each drug type (with and without electronics). Furthermore, requirements of clean-room operations and sterilisation using steam potentially present problems if sensor electronics and batteries are to be fitted in the cartridge assembly prior to cartridges being exerted to steam sterilization and leaving the clean-room production facility.

The sensor unit of the plug unit may include a storage to provide logging of the positional data as one or more events, wherein the or each event is logged together with a time value.

The logging will typically be in the form of a number of events comprising data representing a dose amount in combination with a time value. In other embodiments the logging will be in the form of a number of events comprising data representing only the occurrence of a displacement in combination with a time value, i.e. each event not representing the dose amount but only signifying that a dose expelling has been performed at a given time. The stored data may be in the form of axial or rotation position data only, this allowing the receiving unit, e.g. a smartphone or PC, to calculate the actual drug dose amounts based on supplied information in respect of the type of drug, type of cartridge, and type of device. Alternatively, the logging of the electronic sensor unit may be pre-programmed to work only with a given drug in a given device. Correspondingly, the stored time data may be a relative time indication which is then translated into absolute time in the receiving device.

The circuitry of the electronic sensor unit will typically comprise a processor in the form of a microprocessor, microcontroller or CPU which may be of a general purpose design or be specifically designed for the actual device. The electronic sensor unit may further comprise communication means, such as wired or wireless communication circuitry, for transmitting sensed position data, either when prompted to transmit data, or upon a change in position data of the piston assembly is sensed.

In some embodiments, the cartridge body is formed as a generally tubular shaped body that extends along a central axis.

The proximally arranged thrust receiving surface of the piston assembly may include a proximally facing surface. The proximally facing surface may in some embodiments include a centrally arranged surface feature which is configured for cooperating with a distal end portion of the piston rod of the drug delivery device in a manner so that the distal end of the piston rod aligns centrally when the piston rod exerts a thrust force onto the thrust receiving surface. In some embodiments, the centrally arranged surface feature comprises a protrusion that cooperates with a mating depression arranged on the piston rod at a distal facing surface thereof. In other embodiments, the centrally arranged surface feature comprises a depression, such as a central recessed area, that cooperates with a mating protrusion arranged on the piston rod at a distal facing surface thereof. In other embodiments, the piston assembly includes no centrally arranged surface feature. In some embodiments, the piston assembly has a planar thrust receiving surface.

In some embodiments the piston second member and/or the plug unit defines said thrust receiving surface.

In some systems, the plug unit with the electronic sensor unit is configured to define a self-contained sensor unit.

The electronic sensor unit may in some variants comprise at least one of an ultrasonic sensor and an optical sensor.

In some embodiments the plug unit may comprise a rotating element which couples rotationally to the piston rod in a manner so as to allow the rotating element to rotate as the piston rod rotates during drug expelling. The electronic sensor unit may be configured as a rotary encoder adapted to monitor rotation of the rotary element relative to the piston second member to detect movement of the piston assembly.

In alternative embodiments the rotary encoder comprises an optical sensor arrangement comprising a light source and a light sensor, wherein the optical sensor is arranged in either the rotating element or a non-rotating part of the piston assembly and wherein the other of the rotating element and the non-rotating part of the piston assembly comprises a series of circumferentially disposed reflector surfaces disposed at varying axial positions, each reflector surface being configured for reflecting light emitted by the light source towards the light sensor, and wherein the rotary encoder is configured to sense the distance from the optical sensor to the reflector surface as the rotating element rotates to detect movement of the piston assembly.

In some embodiments the plug unit defines the rotating element. The piston second member and/or the piston first member may be provided with markings which the electronic sensor unit monitors as the plug unit rotates relative to the remainder of the piston assembly.

In other embodiments the plug unit may define a first plug unit part that couples to the piston second member to prevent relative rotation and a second plug unit part that couples rotationally to the piston rod to prevent relative rotation. One of the first plug unit part and the second plug unit part may comprise a plurality of alternating marked areas whereas the other of the first plug unit part and the second plug unit part comprises a sensor configured to sense the plurality of alternating marked areas upon relative rotation between the first plug unit part and the second plug unit part so as to detect movement of the piston assembly.

In some embodiments of the cartridge system the proximally facing opening of the piston first member extends fully to the piston first member distal end, and wherein the piston second member comprises a distally facing wall portion made of a translucent material, the distally facing wall portion being in contact with the drug, and wherein the electronic sensor unit of the plug unit comprises an optical sensor arrangement wherein light impinging from the interior of the cartridge body is transmitted through the distally facing wall portion and received by the optical sensor arrangement.

The cartridge system may be configured to include a at least one reflector surface arranged inside the distal portion of the cartridge body and the optical sensor arrangement may be configured to include a light source and a light sensor. In such system the light sensor may be configured to receive light emitted by the light source and reflected by the reflector surface.

Some embodiments define a cartridge system as defined above in combination with a drug delivery device, wherein the cartridge system is held by the drug delivery device and wherein the drug delivery device comprises a drug expelling mechanism comprising an axially displaceable piston rod configured to provide axial thrust onto the proximally arranged thrust receiving surface of the cartridge system to thereby expel drug from the cartridge.

In some embodiments, the piston rod comprises a thread which mates and engages a thread of another component of the drug delivery device, such that as the expelling mechanism operates, the piston rod travels axially in the distal direction in accordance with the threaded connection.

In further embodiments the drug delivery device comprises a reflector surface arranged axially fixed relative to the cartridge body. In such embodiment, the electronic sensor unit of the piston assembly may be configured to detect axial distance from the reflector surface.

In a second aspect, the present invention relates to a method of forming a cartridge system, the method comprising the steps of:

a) providing a pre-filled cartridge comprising:
a cartridge body accommodating a drug,
a piston slidably disposed in the cartridge body and configured for providing a proximal seal, the piston comprising:
   a piston first member comprising a first material and forming a distal end, a proximal end and a sealing periphery in contact with the cartridge body, wherein the distal end is in contact with the drug and wherein the proximal end of the piston first member includes a proximally facing opening, and
   a piston second member comprising a material of lower compressibility compared to the first material, the piston second member comprising a sleeve-formed portion arranged in the proximally facing opening of the piston first member, wherein the piston second member comprises a proximally facing opening forming a socket,

b) sterilizing the pre-filled cartridge,

c) providing a plug unit coupleable into the socket of the piston second member, wherein the plug unit comprises an electronic sensor unit configured to determine the axial position of the piston within the cartridge body, and

d) subsequent to step b) coupling the plug unit into the socket of the piston second member to form a piston assembly so that the plug unit is at least partially received within the proximally facing opening of the piston first member, and wherein the piston assembly defines a proximally arranged thrust receiving surface configured for receiving a thrust force from a piston rod of a drug delivery device.

In accordance with an embodiment of the second aspect, the cartridge system is formed so that the plug unit is being coupled with the socket of the piston second member only after the piston has been arranged within the cartridge body.

In a further embodiment of the method according to the second aspect, in step c) of providing the plug unit, the method comprises the steps of providing first and second variants of the plug unit, wherein the first variant comprises an electronic sensor unit configured to determine the axial position of the piston within the cartridge body, and wherein the second variant does not include electronic components, such as active electronic components, and wherein, prior to step d), a step of selecting one of the first variant and the second variant for being coupled to the piston second member is performed, and in step d), coupling the selected one of the first variant and the second variant into the socket of the piston second member.

In a third aspect, the invention relates to a method including a cartridge system formed according to the method according to the second aspect, wherein the method further comprises the steps of:
providing a drug delivery device adapted to receive the pre-filled cartridge, and comprising drug expelling means including a piston rod,
providing the pre-filled cartridge,
arranging the pre-filled cartridge to be held by the drug delivery device.

In some embodiments, the cartridge body is formed as a generally tubular shaped body that extends along a central axis.

The proximally arranged thrust receiving surface of the piston assembly may include a proximally facing surface. The proximally facing surface may in some embodiments include a centrally arranged surface feature which is configured for cooperating with a distal end portion of the piston rod of the drug delivery device in a manner so that the distal end of the piston rod aligns centrally when the piston rod exerts a thrust force onto the thrust receiving surface. In some embodiments, the centrally arranged surface feature comprises a protrusion that cooperates with a mating depression arranged on the piston rod at a distal facing surface thereof. In other embodiments, the centrally arranged surface feature comprises a depression, such as a central recessed area, that cooperates with a mating protrusion arranged on the piston rod at a distal facing surface thereof. In other embodiments, the piston assembly includes no centrally arranged surface feature. In some embodiments, the piston assembly has a planar thrust receiving surface.

In some embodiments, the piston rod comprises a thread which mates and engages a thread of another component of the drug delivery device, such that as the expelling mechanism operates, the piston rod travels axially in the distal direction in accordance with the threaded connection.

It is further to be noted that any individual feature or combination of features noted above in accordance with each of the first aspect, the second aspect and the third aspect may be combined with individual features or combination of features according to one or more other of the said first, second and third aspects in agreement with the teaching provided within this disclosure in its entirety.

As used herein, the term “drug” is meant to encompass any flowable medicine formulation capable of being passed through a delivery means such as a cannula or hollow needle in a controlled manner, such as a liquid, solution, gel or fine suspension, and containing one or more drug agents. The drug may be a single drug compound or a premixed or co-formulated multiple drug compounds drug agent from a single reservoir. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid (dispensed) or liquid form. In the description of the exemplary embodiments reference will be made to the use of insulin and GLP-1 containing drugs, this including analogues thereof as well as combinations with one or more other drugs.

### BRIEF DESCRIPTION OF DRAWINGS

In the following the invention will be further described with reference to the drawings, wherein

FIG. 1 shows a perspective view of an example drug delivery pen 100 suitable for use with the present invention,

FIG. 2 shows a partly cut perspective view of distal and proximal sub-assemblies of injection device 100 before final assembly,

FIG. 3a shows a cross-sectional perspective view of main components of a first embodiment of a cartridge system according to the invention having a piston sub-assembly 250′,

FIG. 3b depicts schematically the filling process of the cartridge system of FIG. 3a,

FIG. 4 shows schematically a plug unit for cooperation with the piston sub-assembly 250′ of FIG. 3a,

FIG. 5 shows schematically a cartridge system of the invention and the method of forming variants of a drug delivery pen,

FIG. 6 shows a drug delivery pen provided with an electronic sensor unit and in communication with a smartphone,

FIG. 7 shows schematically the principle of an optical distance meansurement of an electronic sensor unit,

FIG. 8 shows details of a second embodiment of the piston assembly 250 with an electronic sensor unit incorporating an optical sensor,

FIG. 9 shows a perspective partly cut view of an injection pen 100′ having the electronic sensor unit of FIG. 8 incorporated in piston assembly 250,

FIG. 10 shows details of a third embodiment of the piston assembly 250 with an electronic sensor unit incorporating an optical sensor,

FIG. 11 shows a perspective partly cut view of an injection pen 100′ having the electronic sensor unit of FIG. 10 incorporated in piston assembly 250,

FIG. 12 shows details of a fourth embodiment of the piston assembly 250 with an electronic sensor unit incorporating an ultrasonic sensor,

FIG. 13 shows details of a fifth embodiment of the piston assembly 250 with an electronic sensor unit incorporating an ultrasonic sensor,

FIG. 14 shows details of a sixth embodiment of the piston assembly 250 with an electronic sensor unit incorporating an ultrasonic sensor,

FIG. 15 shows details of a seventh embodiment of the piston assembly 250 with an electronic sensor unit incorporating an rotational encoder,

FIGS. 16a and 16b show two methods for forming two different embodiments of piston sub-assemblies 250′ suitable for inclusion in a piston assembly 250 configured as a rotational encoder,

FIG. 17 shows an eight embodiment of the piston assembly 250 with an electronic sensor unit incorporating a stepped rotational encoder,

FIG. 18 shows details of the eight embodiment of the piston assembly 250 with an electronic sensor unit incorporating a stepped rotational encoder, and

FIG. 19 shows a perspective partly cut view of an injection pen 100′ having the electronic sensor unit of FIG. 18 incorporated in piston assembly 250.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DESCRIPTION

In the context of the present disclosure it may be convenient to define that the term “distal end” in the appended figures is meant to refer to the end of the drug delivery device which usually carries the injection needle whereas the term “proximal end” is meant to refer to the opposite end of the drug delivery device pointing away from the injection needle. The shown figures are schematical representations for which reason the configuration of the different structures as well as the relative dimensions are intended to serve illustrative purposes only.

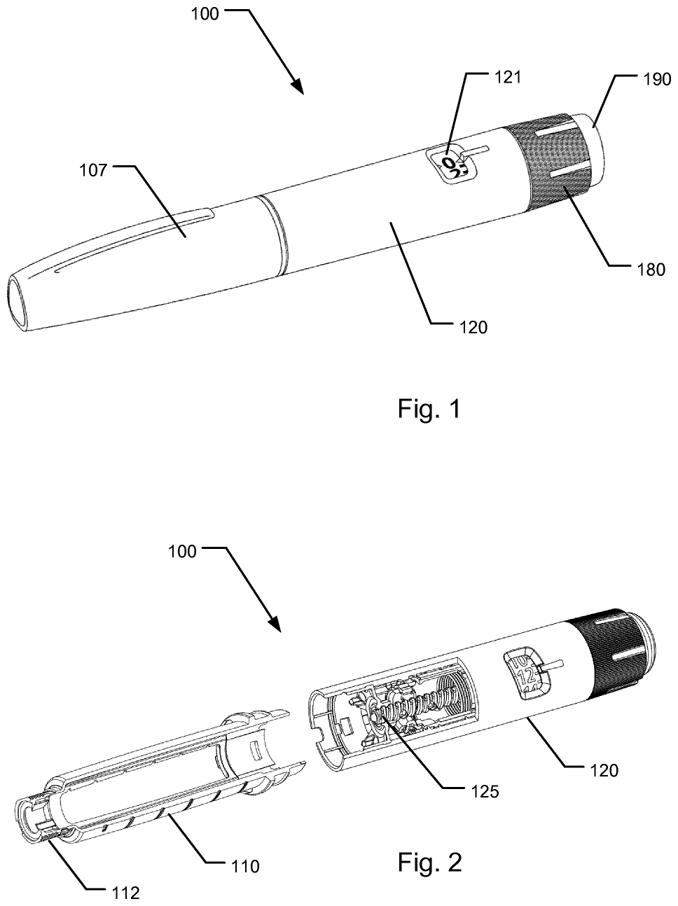

Referring to FIG. 1 an example drug delivery device 100, i.e. a so-called “injection pen” will be described. FIG. 2 shows the drug delivery device 100 in a near-final assembly state, i.e. in a state before a cartridge is inserted in distal cartridge holder 110 and before the cartridge holder with the held cartridge is mounted to the proximal housing 120. The exemplary embodiment of drug delivery device 100 may be configured corresponding to the general design of the device shown in FIGS. 2-5 in WO 2014/161952.

More specifically, the pen device 100 comprises a cap part 107 and a main part having a proximal body or drive assembly portion with a housing 120 in which a drug expelling mechanism is arranged or integrated. The cap part 107 is removably attachable relative to the housing 120. A distal cartridge holder portion 110 becomes revealed upon removal of cap part 107. The distal cartridge holder portion 110 mounts relative to housing 120 and retains a drug-filled transparent cartridge with a distal needle-penetrable septum in a manner so that openings formed in the cartridge holder 110 allows a portion of the cartridge to be inspected. The cartridge is provided with a piston or piston assembly driven along a longitudinal axis by a piston rod 125 forming part of the drug expelling mechanism and may for example contain an insulin, GLP-1 or growth hormone formulation. A proximally arranged rotatable dose dial member 180 serves to manually set a desired dose of drug shown in display window 121 and which can then be expelled when the release button 190 is actuated. Depending on the type of expelling mechanism embodied in the drug delivery device 100 the expelling mechanism may comprise a spring which is strained during dose setting and then released to drive the piston rod 125 when the release button 190 is actuated. Alternatively, the expelling mechanism may be fully manual in which case the dose ring member and the release button move proximally during dose setting corresponding to the set dose size, and then moved distally by the user to expel the set dose. The amount of drug expelled from the cartridge corresponds to the set dose amount as dialled by operating dial member 180.

In the shown example, the piston rod 125 is provided with a thread which mates and engages a thread of another component fixedly associated with the housing so that as the expelling mechanism rotates the piston rod when the release button is actuated, the piston rod travels axially in the distal direction in accordance with the threaded connection and in accordance with the set dose. In other examples, the expelling mechanism may be designed differently so that the piston rod 125 either rotates or is maintained rotationally fixed as the piston rod is being driven axially in the distal direction.

The embodiment shown in FIGS. 1 and 2 show a drug delivery device of the “disposable” or “pre-filled” type, i.e. it is supplied with a pre-mounted cartridge and is to be discarded when the cartridge has been emptied. In alternative embodiments, still in accordance with the present invention, the drug delivery device may be designed to allow a held but emptied cartridge to be replaced, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

In the shown embodiment the cartridge or the cartridge holder is provided with distal coupling means in the form of a needle hub mount having, in the shown example, an external thread 112 adapted to engage an inner thread of a corresponding hub of a needle assembly. In alternative embodiments the thread may be combined with or replaced by other connection means, e.g. a bayonet coupling. The cartridge holder 110 is adapted to receive and hold the cartridge in a loaded position by insertion of the cartridge in a distal direction relative to the cartridge holder. The holder has a generally tubular configuration with a distal retaining region adapted to axially receive the distal end of the cartridge. In alternative embodiments, the cartridge may include other delivery members than a needle assembly. Still alternatively the delivery member in form of an injection needle may be fixedly attached to the cartridge body, e.g. forming a pre-filled syringe.

During assembly, after a drug-filled cartridge has been inserted into the cartridge holder, the cartridge holder 110 is attached to the housing 120. Typically, as utilized in the shown embodiment, a permanent attachment is provided, such as by a snap connection, between the cartridge holder 110 and the housing 120 so that once the attachment has been obtained the cartridge holder cannot subsequently be separated from the housing.

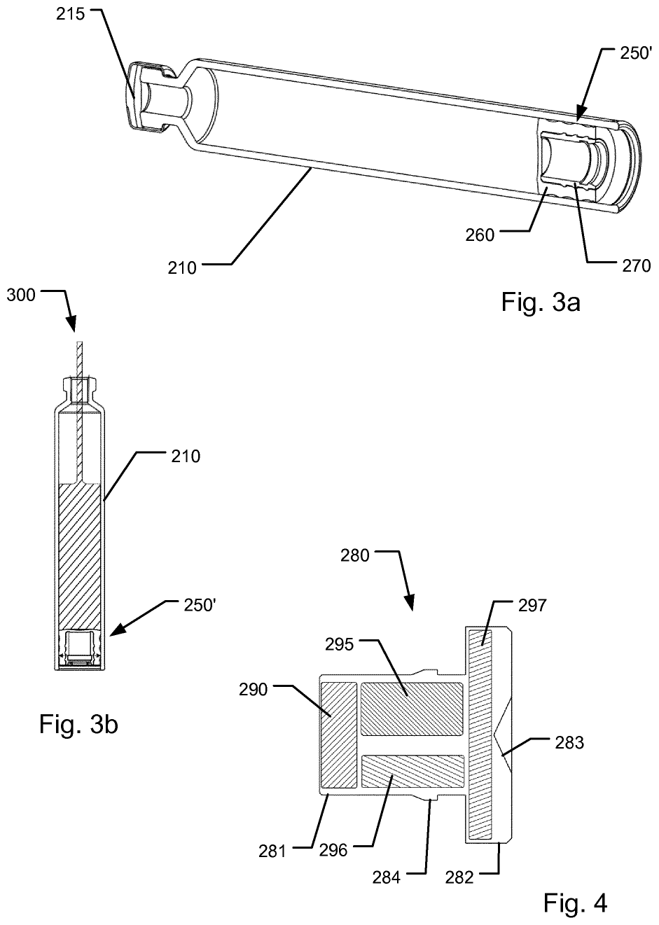

FIG. 3a shows main components of a first embodiment of a cartridge system according to the invention. The main components include a generally cylindrical cartridge body 210 extending along a central longitudinal axis and having a distal neck portion defining an outlet of the cartridge body. In the shown embodiment, the cartridge body is made of glass and the outlet is closed by a pierceable septum 215 which is held in place by a cap. The pierceable septum 215 is adapted for being pierced by a needle assembly for establishing fluid communication with the interior of the cartridge when the needle assembly is connected to the drug delivery device 100.

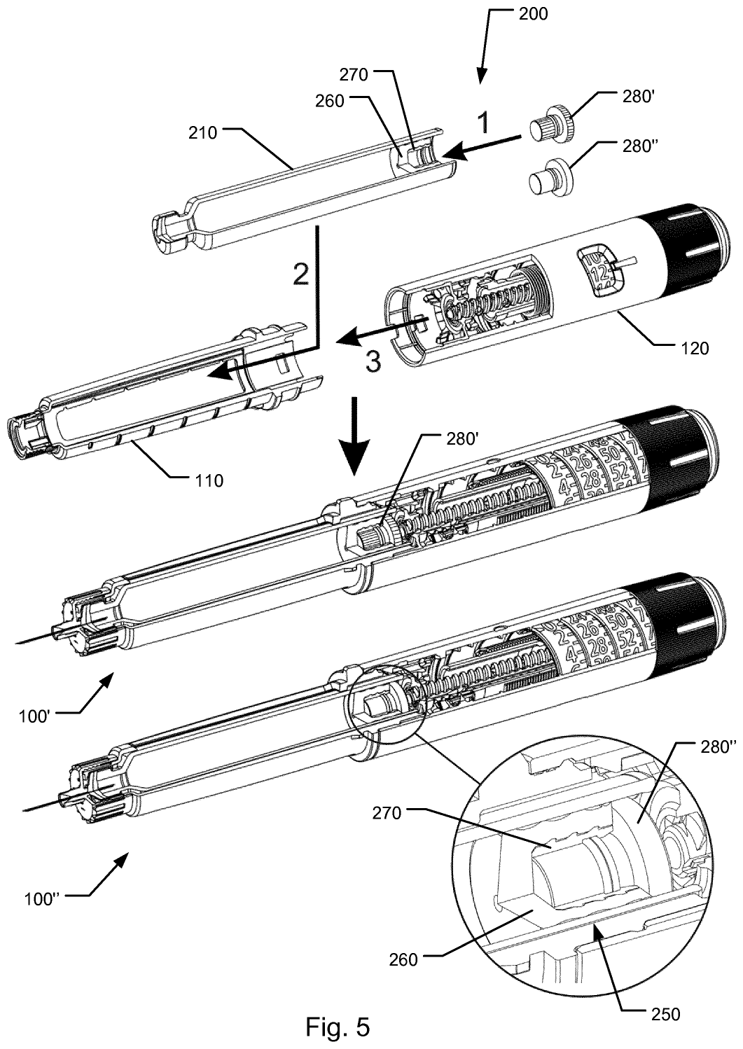

The cartridge body 210 is sealed proximally by a slideably arranged piston assembly 250. The piston assembly 250 is configured to be driven axially in the distal direction to expel one or more drugs accommodated between the piston assembly 250 and the outlet of the cartridge. In FIG. 3a, only a first subset of components for piston assembly 250 is shown and reference is made to FIG. 5 which shows an example of all components of a piston assembly 250 arranged internally in a cartridge system in accordance with the invention. FIG. 3a shows a piston sub-assembly 250′ which is arranged inside the cartridge body 210 in a position representing an initial position prior to drug expelling, e.g. in proximity of the proximal end of the cartridge body. As indicated in FIG. 3a the piston sub-assembly 250′ comprises a piston first member 260 and a piston second member 270. In the shown embodiment the piston first member 260 is formed as a generally tubular member that has a distal end, a proximal end and a sealing periphery between the distal and proximal ends, wherein the sealing periphery is in contact with the interior wall surface of the cartridge body 210. The distal end is in contact with the medicament. The proximal end of the piston first member 260 includes a proximally facing opening leading to a recessed area. The piston first member is made from a first material which typically will be a soft, compliant material, such as an elastomeric material, in order to have the required properties to sealingly yet slideably adhere to the cartridge body interior wall.

The piston second member 270 made from a material of lower compressibility compared to the first material. Suitable non-limiting examples of materials for the piston second member may include plastic materials, such a thermoplastic polymer. The piston second member 270 comprises a sleeve-formed portion arranged in the proximally facing opening of the piston first member. The piston second member 270 further comprises a proximally facing opening leading to a cavity that forms a socket. As will be described later, the socket is configured to receive different variants of a plug unit 280, where the plug unit may either take the form of an electronic sensor unit or take the form of a dummy plug unit which do not incorporate electronic components. In the shown embodiment the piston second member 270 is formed as a tubular sleeve having a generally cylindrical outer surface and a generally cylindrical inner surface. In the shown embodiment the cavity of the piston second member 270 leads to an open distal end. In other embodiments which will be described later the sleeve may alternatively be formed with a closed end wall at the distal end of the cavity. Also, in other embodiments, instead of the sleeve having generally cylindrical inner and outer surfaces, the inner and outer surfaces may be formed having other shapes than cylindrical. As will be described later, the interface between the piston second member 270 and the piston first member may include cooperating geometries to ensure that the piston second member 270 is retained fixedly within the piston first member 260, optionally in a fluid-tight manner. As the piston second member 270 is made from a rigid material the piston second member provides stability to the piston first member 260 so that the piston 250 will obtain optimal sealing properties.

FIG. 3b depicts schematically the filling step of a cartridge wherein, during filling of the cartridge with drug 300, the cartridge body 210 is sealed at the proximal end only by the piston sub-assembly 250′ and therefore the piston second member 270 is required for providing structural rigidity to the piston first member 260 if a significant cavity is to be made in the piston first member.

FIG. 4 shows a schematic representation of a plug unit 280 for cooperation with the piston sub-assembly 250′ of FIG. 3a, the plug unit 280 comprising an integrated electronic unit. The illustration is not depicting actual components or their expected size or location within the integrated electronic sensor unit, but only represents key components and functional features of the electronic unit.

The plug unit 280 is formed to become at least partly received into the proximal cavity of the piston second member 270 and couple thereto to be retained therein. In order to ensure relative fixation, the plug unit 280 and the piston second member 270 may comprise corresponding engaging geometries such as a threaded fitting or a snap fitting. In the shown embodiment a radially protruding ring 284 is provided to be received into a ring-shaped radial recess of the piston second member 270. The plug unit 280 shown comprises a sensor 290, electronic circuitry with means of data processing 295, wireless communication unit 296 and a power source such as a battery 297. As will be described later, the plug unit may comprise an electronic sensor unit configured to determine the axial position within the cartridge body 210.

The shown plug unit 280 is formed with a distal portion 281 having a tubular outer shape with a diameter sized to fit into the socket of piston second member 270 and a proximal portion 282 forming a rim section that has a somewhat larger diameter but slightly smaller than the internal diameter of cartridge body 210. The proximal portion is located proximally relative to both the piston first member 260 and the piston second member 270 and radially outside the socket of the piston second member 270 when the distal portion 281 of the plug unit 280 is received in fitting relationship within the socket. In the shown embodiment the proximal end face of the plug unit 280 includes a central recessed area 283 providing a receiving surface for the distal end of the piston rod 125 of the expelling mechanism. The shape of the receiving surface may differ from the shown shape but will be formed for receiving a thrust force from the piston rod of the drug delivery device in a manner to ensure a self-centering effect. The different components 290, 295, 296 and 297 may be distributed in other ways within the distal portion and proximal portion of the plug unit 280 than shown in FIG. 4. Also, in other embodiments, the shape of the plug unit 280 may be different. For example, the plug unit 280 may be sized and formed to be fully accommodated within the socket of the piston second member 270.

FIG. 5 depicts schematically a cartridge system of the invention and the steps of initially forming a cartridge 200 and subsequently forming a sample drug delivery device 100 in two different variants. In step 1 a plug unit 280 is selected by either selecting a plug unit 280′ having an electronic sensor unit, or alternatively selecting a “dummy” plug unit 280″ having no electronic components. The selected plug unit 280′, 280″ is inserted into the piston sub-assembly 250′ of the cartridge so that the selected plug unit snaps into the piston second member 270 to become fixedly received therein. Hence, a piston assembly 250 is formed inside cartridge 200. The cartridge thus formed is in step 2 inserted into the cartridge holder 110. In step 3, the cartridge holder is fixedly attached to the housing 120. Depending on the variant of the plug unit, i.e. 280′ or 280″, the final drug delivery device will either form an electronically enabled drug delivery device 100′ or a simpler drug delivery device 100″ not containing an electronic sensor unit. For both variants, a needle assembly has been shown attached to each of the drug delivery devices. However, in other embodiments, a needle assembly is only connected upon use of the device. Regardless of the variant of drug delivery device, the final device is ready for labelling and packaging.

In accordance with an aspect of the invention the overall functionality of the electronic sensor unit contained within a plug unit 280 is to detect use/out-dosing, measure the size of the dose being administered by means of the drug delivery pen 100′ and transmit the time and size of one or more of the recently expelled doses by means of wireless communication to an external device for logging and displaying data.

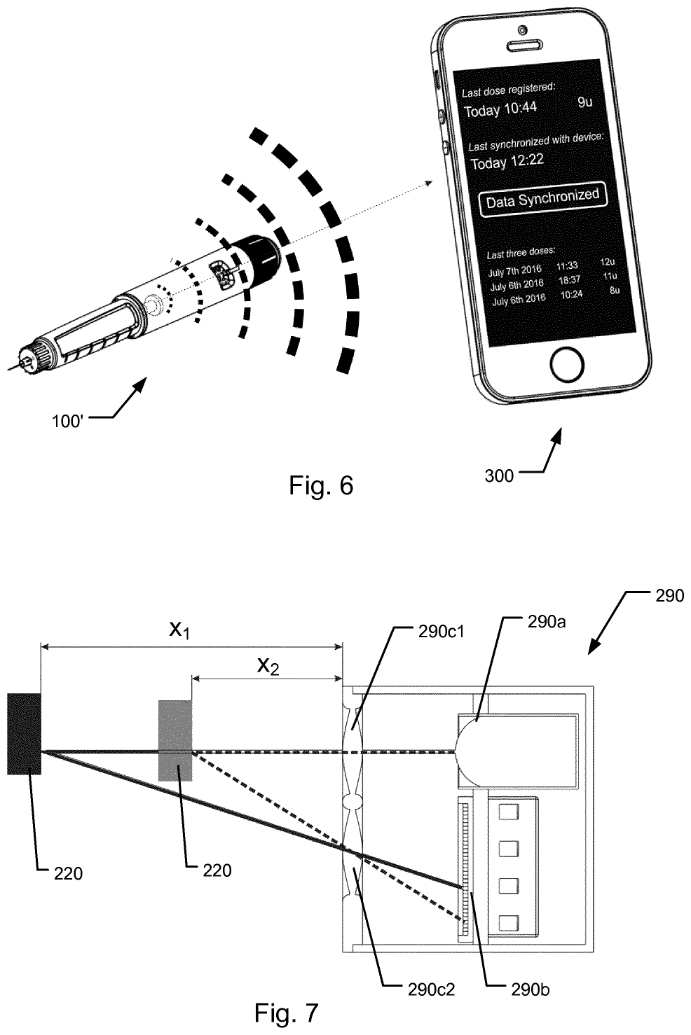

FIG. 6 shows a drug delivery pen 100′ provided with an electronic sensor unit, the pen being arranged next to a smartphone 300 configured to receive logging data from the electronic sensor unit together with associated data via wireless communication, e.g. NFC, Bluetooth etc.

In order to communicate with the electronic sensor unit the smartphone 300 has been provided with specific “insulin diary” software. When the software is activated to initiate data transfer the smartphone NFC transmitter will transmit specific code which will wake up any nearby electronic sensor unit which will then retransmit a unique code identifying the specific module. The sensor unit may transmit information in respect of the drug in the cartridge. In this way the smartphone can create an insulin diary and indicate the specific drug. In the shown embodiment log data from an electronic sensor unit associated with the particular drug delivery device 100′ has been transferred. In the exemplary user interface a view is being offered showing the different amounts of drug delivered together with a real time value for a number of recent dose deliveries.

In the following different aspects concerning sensor types and socket designs are described. The purpose of the sensor is to determine the position or movement of the piston assembly 250 and thereby be able to determine the volume expelled, based on knowledge of the dimensions of the cartridge. This can be achieved using a number of different sensor types and principles.

The movement of the piston assembly 250 in a given out-dosing can be determined either as a relative change where the sensor is monitored and a number of “counts”, from a change is detected to no change can be detected, are registered to determine the axial distance of piston assembly movement. This option however presents the challenge that a change must be registered, which means that the system has to wake up prior to out-dosing being activated or be monitoring constantly (which increase power consumption significantly), to ensure detection of initial change in piston assembly position.

A better solution is to measure an absolute position and store this. This will provide a reference to which a suspected change can be compared and allow some additional features to be incorporated, such as display of remaining volume and counterfeit warning. Movement can be determined as the difference between the present detected stop position minus last detected stop position. Counterfeit/reuse will result in a negative difference during first use and thus be easily detected.

The axial position of the piston assembly can be determined directly by measurement of the distance from a given reference point or surface. This reference point or surface can be arranged in the distal end of the cartridge and would thus require measurement through the fluid in front of the piston assembly, thereby measuring a decreasing distance as the piston assembly moves forward during out-dosing. The reference point or surface can also be placed in the direction opposite the cartridge container, i.e. arranged proximally to the piston assembly whereby measured distance will increase as the piston assembly moves forward during out-dosing. In devices where the piston assembly is actuated by a threaded piston rod driven by rotation through a nut, the axial movement can also be determined from measurement of the rotation of the piston rod and knowledge of the inclination of the piston rod threading. In the following the working principle of some relevant sensor types and examples of application of such sensors is provided.

Optical distance measurement sensors measures distances by one of three different methods. The simple time-of-flight is based on emitting a light pulse and measuring the time of the reflected light to arrive. Since the speed of light is very high, this method is best suited for larger distances and will most likely not be suitable for use in this context. Likewise, distance measurement based on phase shift measurement will most likely be less suitable in this context, due to the limited accuracy when measuring very small distances and limited resolution.

The third method of measuring distances using an optical measuring device is based on emitting a light beam and detecting its reflection through a lens (that may increase the angle of refraction) onto a CCD chip with an array of sensors. By detecting the location on the CCD-chip array the refracted light impinges, the incoming angle can be determined and from knowledge of the (fixed) distance between light source and CCD-chip, an accurate distance can be calculated by triangulation. Such sensors are readily and cheaply available today.

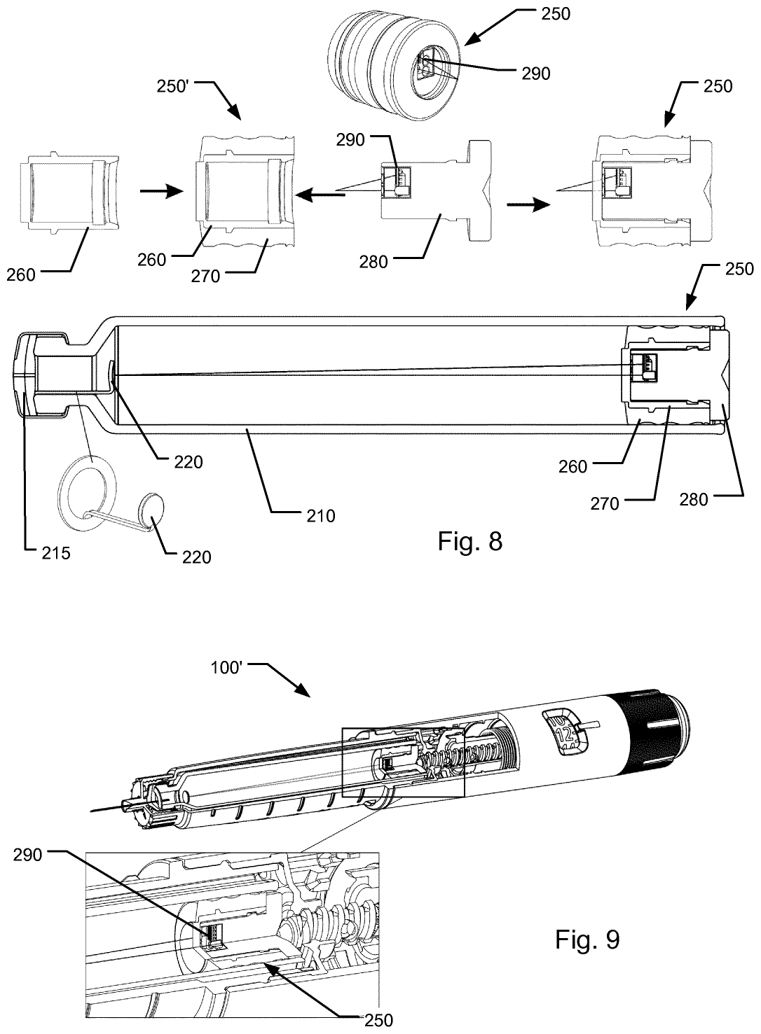

FIG. 7 schematically illustrates the working principle for an Optical Distance Measurement sensor (ODM sensor). The electronic sensor unit in this example includes a sensor 290 that comprises the following components. In FIG. 7 a light source 290a, such as a laser or LED, is arranged in a light source housing. A light sensor array 290b, such as CCD-chip or a series of photo-sensors, is arranged laterally with respect to the light source 290a. A lens 290c1 and 290c2 is arranged in front of each of the light source 290a and the light sensor array 290b. Referring to FIG. 8, lower section which depicts a cross-sectional side view of a cartridge system, a reference reflector surface 220 is arranged at the distal end of cartridge body 210 at a central location and axially at a position where it will not interfere with an attached needle assembly. In the shown embodiment, as indicated, the reference reflector surface 220 includes structure for mounting the reference reflector surface relative to the outlet of the cartridge.

In FIG. 7 light emitted from the light source 290a is transmitted through lens 290c1 to impinge on reference reflector surface 220 arranged at the distal end of the cartridge. Light reflected from the reference reflector surface 220 is transmitted through lens 290c2 and is picked up by the light sensor array 290b. The reference reflector surface 220 is shown in FIG. 7 at two different axial locations X₁ and X₂, each location representing the piston assembly assuming a particular distance from the outlet of the cartridge and thus from the reference reflector surface 220. As indicated the location of the reflected ray of light impinging on the light sensor array 290b is dependent on the axial distance between the light source 290a and the reference reflector surface 220 and the position or movement determining circuitry of the electronic sensor unit of the piston assembly 250 utilizes this principle in determining the expelled dosage from the cartridge.

FIG. 8 illustrates details of the piston assembly 250 of a second embodiment of the cartridge system utilizing the principle shown in connection with FIG. 7. In order to enable light transmission in a distal direction from the electronic sensor unit of the plug unit 280 to the reference reflector surface 220, and to receive light reflected from the reference reflector surface, the electronic sensor unit is arranged in the socket of piston second member which is made from a transparent material, or made of a material penetrable to light of the wavelength used by the sensor. The piston first member 260 is made with an axial through-going opening meaning that the distal part of the piston first member includes a distal opening leading to the internal cavity. As depicted in FIG. 8 the piston second member 270 is arranged inside the through-going opening of the piston first member 260. In this way the piston second member 270 is exposed to contact with the drug contained in cartridge body 210. The piston second member 270 in the second embodiment is formed as a closed socket with a distal end wall with no openings. By forming the piston first member and the piston second member by a 2-component moulding process, the interface between the components can be made leak-tight and allows the socket component, i.e. the piston second member 270, to protrude through the piston first member 260 without risking leaks, even when the plug unit 280 is not plugged into the socket. Alternatively, the piston second member 270 is formed separately from the piston first member 260 and subsequently joined or assembled in a fluid-tight manner.

FIG. 9 shows a perspective view of a partly cut injection pen 100′ which incorporates the piston assembly 250 and the cartridge according to the second embodiment as described above and shown in FIG. 8.

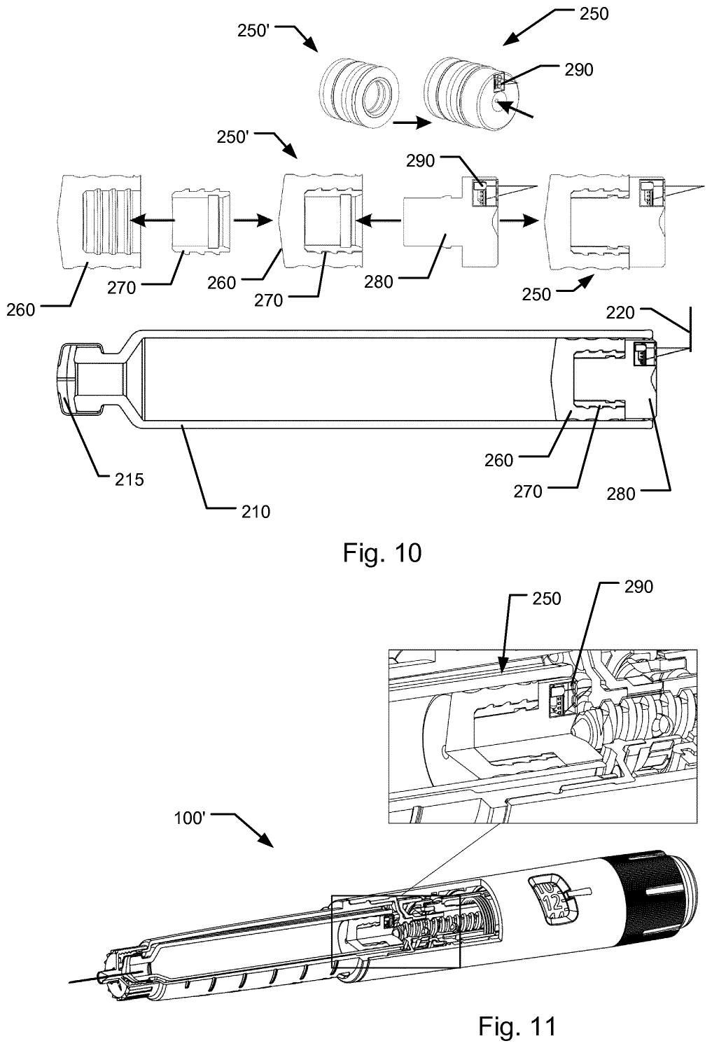

FIG. 10 illustrates details of the piston assembly 250 of a third embodiment of the cartridge system utilizing the principle shown in connection with FIG. 7. The piston sub-assembly 250′ formed by the piston first member 260 and the piston second member 270 correspond to the design described in connection with the first embodiment. In the third embodiment a light sensor is arranged to direct light in the proximal direction relative to the piston assembly 250 towards a reference reflector surface 220 arranged proximal to the cartridge and to receive light reflected from the reference reflector surface.

FIG. 11 shows a perspective view of a partly cut injection pen 100′ which incorporates the piston assembly 250 and the cartridge according to the third embodiment as described above and shown in FIG. 10.

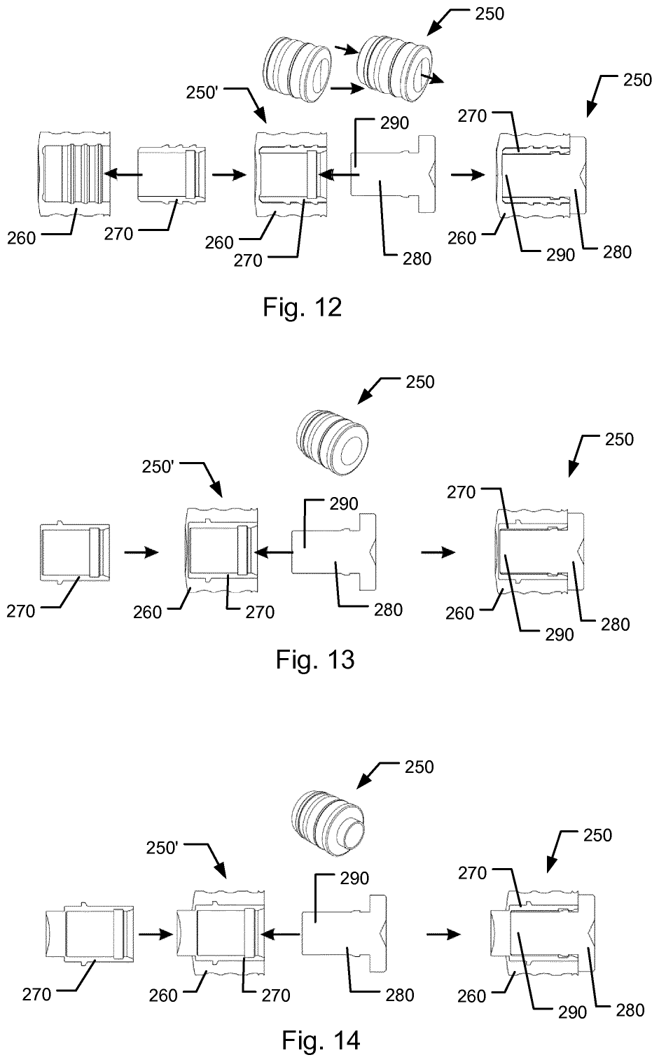

In the fourth through sixth embodiments shown in FIG. 12-14 an ultrasonic sensor 290 is incorporated into a plug unit 280 to determine the position of the piston assembly 250 inside the cartridge. The ultrasonic sensor which incorporates a transducer measures the distance between the front of the transducer and a reflector at the distal end of the cartridge by emitting a signal and monitoring the returning signal. The distance can be calculated from measuring the time difference between the transmitted and received signals and knowledge of the velocity of sound in the fluid. When improving the signal-to-noise ratio in ultrasonic measurements, not only the transmission of sound has to be optimised. The reflection of sound and the ability to distinguish the relevant reference signal is equally important.

The design shown in FIG. 12 is based on an assembly of two components. A rigid, stabilizing socket of a plastic material defining a piston second member 270 is inserted into a piston first member 260 that serves as a sealing member, thus of very flexible material, to form a piston sub-assembly 250′. The socket is open to allow direct contact with an ultrasonic transducer later inserted into the socket. To eliminate the risk of air gaps between the transducer and the piston first member 260, the piston first member is dimensioned so that when the plug unit 280 including the transducer is inserted into the socket the distal material portion of the piston first member 260 is stretched. This will minimise the risk of airgaps preventing the signal from transducer to fluid.

In a fifth embodiment shown in FIG. 13 the design is based on a two-component moulding, in which a closed socket of a rigid material defining a piston second member 270 is first moulded and second more flexible (and thus more dampening) material defining a piston first member 260 is moulded onto the piston second member 270 to provide the sealing capabilities. The two-component moulding eliminates the risk of air gaps between the closed socket formed by the piston second member 270 and the piston first member 260 distal portion and allows for a very thin layer of soft material in front of the socket. Thereby the dampening of the signal can be limited.

In a sixth embodiment shown in FIG. 14 the design is also based on a two-component moulding, but in this design, a delay line is integrated in the socket formed by the piston second member 270. The delay line helps reducing the effects of the transducers nearfield that makes it difficult to detect echoes originating from very close to the transducer. This may become an issue when the piston assembly 250 assumes a position wherein the cartridge is almost empty. The two-component moulding allows for sealing between the sealing part defined by the piston first member 260 surrounding the socket formed by the piston second member 270 and protruding the piston first member 260.

As opposed to the previous embodiments described above which determines the position or the movement of the piston assembly in the cartridge on the basis of measured axial dimensions, the electronic sensor unit of the plug unit in the following embodiments measures the rotational movement of the piston rod of the drug delivery pen during out-dosing. In such embodiments the piston rod moves in a helical movement as defined by a threaded connection in the drug expelling mechanism and, as the axial movement of the piston assembly is correlated with the rotational movement of the piston rod, the axial movement of the piston assembly can be easily deducted. For this purpose, the plug unit comprises a rotating element which couples to the piston rod in a manner so as to allow the rotating element to rotate as the piston rod rotates during drug expelling. The plug unit comprises an electronic sensor unit configured as a rotary encoder which is adapted to monitor rotation of the rotary element relative to the piston second member.

As will be described further below, the plug unit may be provided either as a single-member plug unit, or as a two-member plug unit having two sub-parts that are able to rotate relative to each other.

In a single-member plug unit (not shown in figures), the single-member plug unit couples to the piston rod so as to prevent relative rotation between piston rod and the plug unit. The single-member plug unit includes the electronic sensor unit which is rotated by the piston rod. The piston second member and/or the piston first member may be provided with markings or the like which the electronic sensor unit will monitor as the single-member plug unit rotates relative to the remainder of the piston assembly.

In a two-member plug unit a rotational encoder system can be disposed in one of the subparts, or distributed within the two subparts, to monitor rotational movement between the two sub-parts. In such system, a first one of the subparts couples to the piston second member to prevent relative rotation whereas the other of the subparts couples to the piston rod to prevent relative rotation.

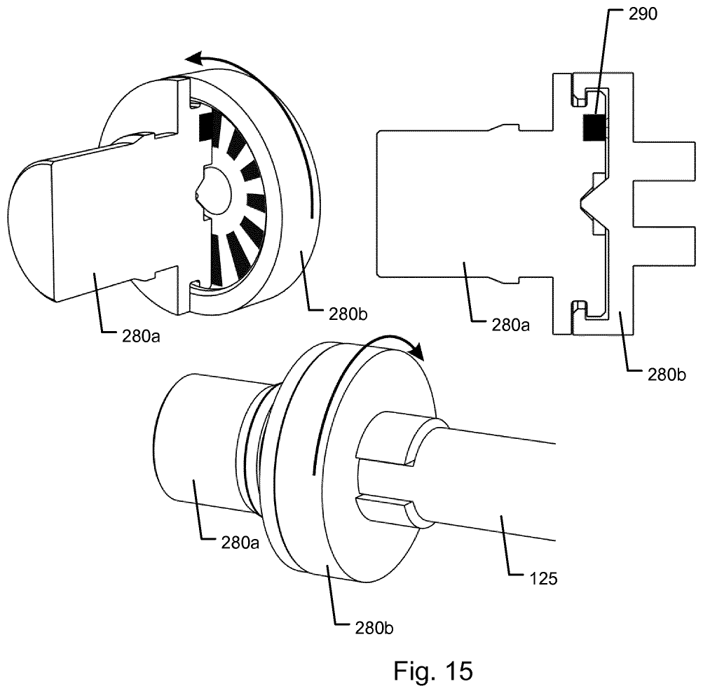

An example of a two-member plug unit is shown in FIG. 15 which forms a seventh embodiment. In this embodiment the two-member plug unit comprises a non-rotating first plug member 280a and a rotating second plug member 280b, i.e. the second plug member 280b is allowed to rotate relative to the first plug member 280a. The second plug member 280b may be configured similar to a piston washer to which the piston rod 125 is connected when the second plug member 280b is inserted into the pen delivery device. In other non-shown embodiments the second plug member may be provided as part of the expelling mechanism of housing 120 so that the second plug member mates and couples with the first plug member as the first plug member is inserted into the pen delivery device. The second plug member 280b connects to the piston rod 125 such that rotation between the second plug member and the piston rod is prevented. In the shown embodiment, the second plug member 280b is attached axially to the first plug member 280a which comprises the electronic unit in a manner so that the second plug member 280b is allowed to rotate relative to the first plug member 280a.

A number of different encoding principles and sensor types can be utilized in order to measure the second plug member's rotation relative to the electronic sensor unit by using the electronic sensor unit of the first plug member 280a. An optical sensor 290 can be incorporated in the first plug member 280a and used to count the number of alternating marked areas on the inner surface of the second plug member 280b passing by the sensor, as illustrated in the upper left part of FIG. 15. The marked areas may be obtained by varying the colour or the reflectivity of the markings. Capacitive sensors, resistive sensors, magnetic sensors and other sensor types may alternatively be used for this purpose.

It is evident that most solutions based on the tracking of the rotation of the piston rod/second plug member 280b and calculation of the position/movement of the piston assembly 250 from the piston rod threading will depend on the number of full revolutions of the rotatable element 280b that is counted during motion. Hence, an absolute axial position determination of the piston assembly 250 cannot be established from the angular position of the second plug member 280b alone.

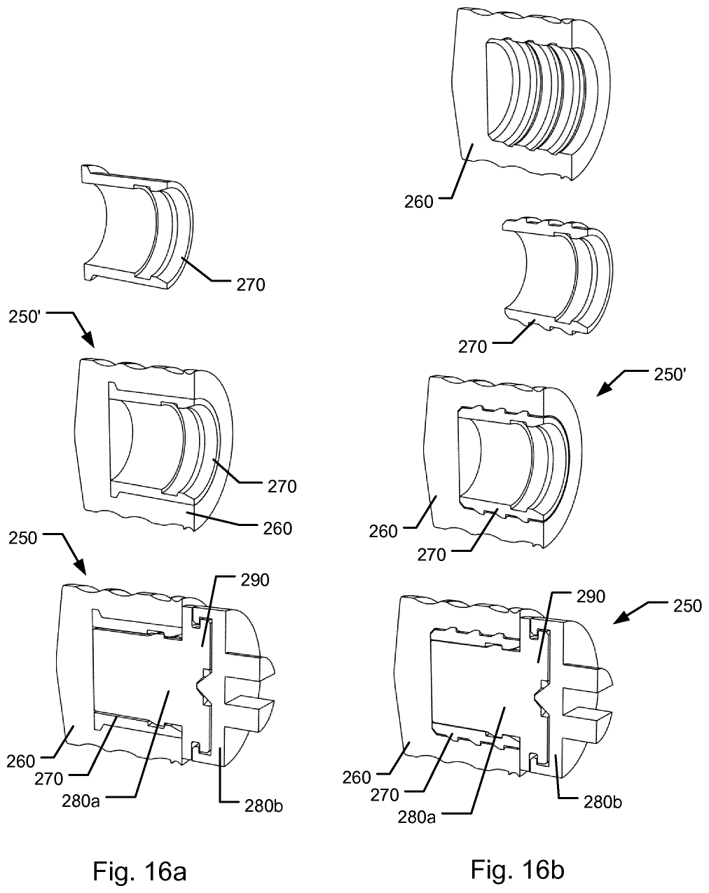

FIGS. 16a and 16b show two methods for forming two different embodiments of piston sub-assemblies 250′ suitable for inclusion in piston assemblies 250 configured as rotational encoders of the type described in connection with FIG. 15. In FIG. 16a a 2-component moulding is used to form a piston sub-assembly 250′ consisting of the piston first member 260 and the piston second member 270 to ensure sealing and prevent rotation between these components. In FIG. 16b the piston first member 260 and the piston second member 270 are formed separately and subsequently joined to form a piston sub-assembly 250′, again in a manner to ensure sealing and prevent rotation between these components. After each of the sub-assemblies 250′ have been formed, the plug unit 280a/280b is plugged into the socket of the piston second member 270 and the resulting piston assembly 250 will be formed. Upon insertion of the cartridge with the piston assembly 250 into a cartridge holder 110 and subsequent coupling with the housing 120 of the pen delivery device coupling geometries of the second plug member 280b is aligned rotationally with cooperating coupling geometries of the piston rod 125 so that the second plug member 280b couples rotationally with the piston rod 125 enabling the piston rod to drive the second plug member for rotation.

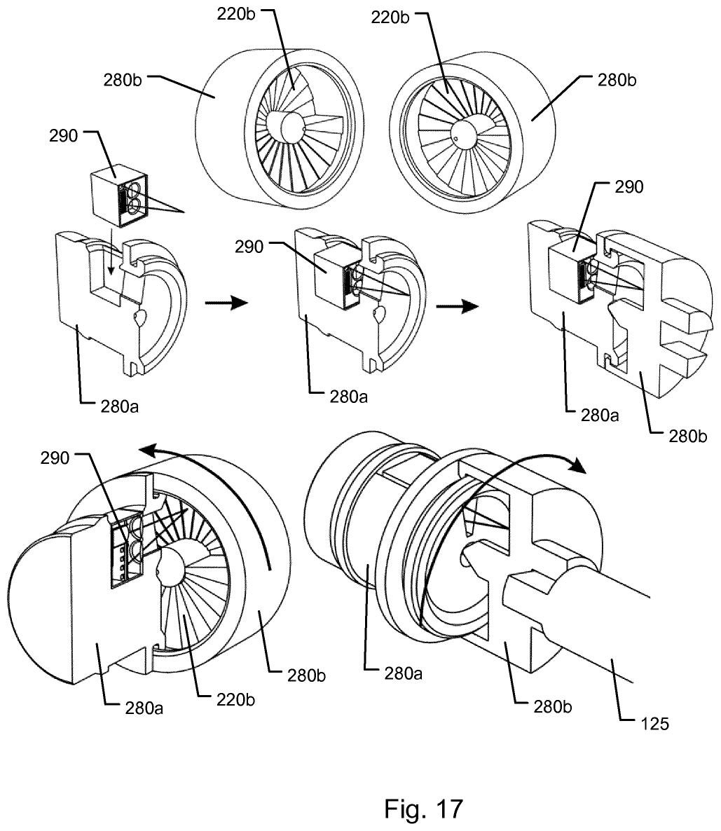

FIG. 17 shows an eight embodiment of the plug unit 280a/280b for a piston assembly 250 with an electronic sensor unit 290 incorporating an optical distance measurement sensor configured as a stepped rotational encoder. The stepped rotational encoder is used to enable determination of absolute angular positioning of the second plug member 280b relative to the first plug member 280a within a single revolution. By designing the second plug member 280b with a plurality of stepped areas 220b arranged angularly with respect to each other and at different axial positions, the optical distance measurement sensor will measure different distances depending on angular orientation of the second plug member 280b relative to the first plug member 280a. The measuring principle for the optical distance measurement sensor may utilize the principle described in connection with FIG. 7.

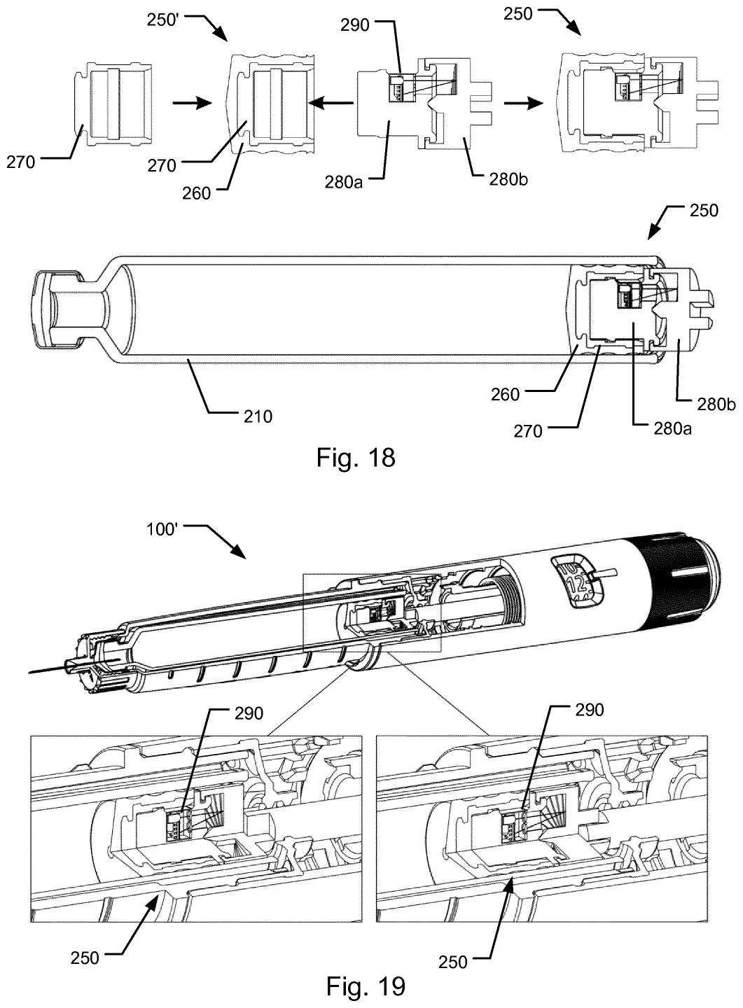

FIG. 18 illustrates details of the piston assembly 250 of the eight embodiment of the cartridge system utilizing the principle shown in connection with FIG. 17. The piston sub-assembly 250′ formed by the piston first member 260 and the piston second member 270 may be made by a 2-component moulding process. After the piston sub-assembly 250′ has been inserted into the cartridge and the cartridge has been filled with a drug, the two-member plug unit 280a/280b is plugged into the socket of the piston second member 270 to provide the piston assembly 250 in the cartridge system. Hereafter, the cartridge system can be coupled with the drug delivery device.

FIG. 19 shows a perspective view of a partly cut injection pen 100′ which incorporates the piston assembly 250 in the cartridge system according to the eight embodiment as described above and shown in FIG. 18.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A cartridge system for use with a drug delivery device the cartridge system comprising:
a pre-filled cartridge comprising:
a cartridge body accommodating a drug,
a piston slidably disposed in the cartridge body and configured for providing a proximal seal, the piston comprising:
a piston first member comprising a first material and forming a distal end, a proximal end and a sealing periphery in contact with the cartridge body, wherein the distal end is in contact with the drug and wherein the proximal end of the piston first member includes a proximally facing opening, and
a piston second member comprising a material of lower compressibility compared to the first material, the piston second member comprising a sleeve-formed portion arranged in the proximally facing opening of the piston first member, wherein the piston second member comprises a proximally facing opening forming a socket; and
a plug unit coupleable or coupled into the socket of the piston second member, wherein the plug unit comprises an electronic sensor unit configured to determine the axial position of the piston within the cartridge body;
wherein the piston with the plug unit coupled into the socket of the piston second member defines a piston assembly wherein the plug unit is at least partially received within the proximally facing opening of the piston first member, and wherein the piston assembly defines a proximally arranged thrust receiving surface configured for receiving a thrust force from a piston rod of the drug delivery device.

2. The cartridge system as defined in claim 1, wherein the piston second member and/or the plug unit defines said thrust receiving surface.

3. The cartridge system as defined in claim 1, wherein the plug unit with the electronic sensor unit defines a self-contained sensor unit.

4. The cartridge system as defined in claim 1, wherein the electronic sensor unit comprises at least one of an ultrasonic sensor and an optical sensor.

5. The cartridge system as defined in claim 1, wherein the plug unit comprises a rotating element which couples rotationally to the piston rod in a manner so as to allow the rotating element to rotate as the piston rod rotates during drug expelling, and wherein the electronic sensor unit is configured as a rotary encoder adapted to monitor rotation of the rotary element relative to the piston second member to detect movement of the piston assembly.

6. The cartridge system as defined in claim 5, wherein the rotary encoder comprises an optical sensor arrangement comprising a light source and a light sensor, wherein the optical sensor is arranged in either the rotating element or a non-rotating part of the piston assembly and wherein the other of the rotating element and the non-rotating part of the piston assembly comprises a series of circumferentially disposed reflector surfaces disposed at varying axial positions, each reflector surface being configured for reflecting light emitted by the light source towards the light sensor, and wherein the rotary encoder is configured to sense the distance from the optical sensor to the reflector surface as the rotating element rotates to detect movement of the piston assembly.

7. The cartridge system as defined in claim 5, wherein the plug unit defines the rotating element and wherein the piston second member and/or the piston first member is/are provided with markings which the electronic sensor unit monitors as the plug unit rotates relative to the remainder of the piston assembly.

8. The cartridge system as defined in claim 5, wherein the plug unit defines a first plug unit part that couples to the piston second member to prevent relative rotation and a second plug unit part that couples rotationally to the piston rod to prevent relative rotation, wherein one of the first plug unit part and the second plug unit part comprises a plurality of alternating marked areas and wherein the other of the first plug unit part and the second plug unit part comprises a sensor configured to sense the plurality of alternating marked areas upon relative rotation between the first plug unit part and the second plug unit part so as to detect movement of the piston assembly.

9. The cartridge system as defined in claim 1, wherein the proximally facing opening of the piston first member extends fully to the piston first member distal end, and wherein the piston second member comprises a distally facing wall portion made of a translucent material, the distally facing wall portion being in contact with the drug, and wherein the electronic sensor unit of the plug unit comprises an optical sensor arrangement wherein light impinging from the interior of the cartridge body is transmitted through the distally facing wall portion and received by the optical sensor arrangement.

10. The cartridge system as defined in claim 9, wherein the cartridge system includes a at least one reflector surface arranged inside the distal portion of the cartridge body, wherein the optical sensor arrangement includes a light source and a light sensor, and wherein the light sensor is configured to receive light emitted by the light source and reflected by the reflector surface.

11. The cartridge system as defined in claim 1 in combination with a drug delivery device, wherein the cartridge system is held by the drug delivery device and wherein the drug delivery device comprises a drug expelling mechanism comprising an axially displaceable piston rod configured to provide axial thrust onto the proximally arranged thrust receiving surface of the cartridge system to thereby expel drug from the cartridge.

12. The cartridge system as defined in claim 11, wherein the drug delivery device comprises a reflector surface arranged axially fixed relative to the cartridge body, wherein the electronic sensor unit of the piston assembly is configured to detect axial distance from the reflector surface.

13. A method of forming a cartridge system according to claim 1, the method comprising the steps of:
a) providing a pre-filled cartridge comprising the cartridge body, the piston first member, the piston second member, and the drug accommodated within the cartridge body,
b) sterilizing the pre-filled cartridge,
c) providing the plug unit,
d) subsequent to step b) coupling the plug unit relative to the piston second member to form a piston assembly, wherein the plug unit is at least partially positioned within the proximally facing opening of the piston first member.

14. The method of forming a cartridge system according claim 13, wherein in step c) of providing the plug unit comprises providing first and second variants of the plug unit, wherein the first variant comprises an electronic sensor unit configured to determine the axial position of the piston within the cartridge body, and wherein the second variant does not include active electronic components, and wherein prior to step d) a step of selecting one of the first variant and the second variant for being coupled to the piston second member is performed.

15. A method of forming a drug delivery device including a cartridge system formed according to the method defined in claim 13, wherein the method further comprises the steps of:
providing a drug delivery device adapted to receive the pre-filled cartridge, and comprising drug expelling structure including a piston rod,
providing the pre-filled cartridge,
arranging the pre-filled cartridge to be held by the drug delivery device.
